(19) United States
(12) Patent Application Publication
(10) Pub. No.: US 2008/0033297 A1
(43) Pub. Date: Feb. 7, 2008

(54) Neural tissue stimulation, assessment, mapping, and therapy utilizing targeted acoustic mechanisms

(76) Inventor: John W. Sliwa, Los Altos, CA (US)

Correspondence Address: David W. Collins;Intellectual Property Law, Suite 100, 512 E. Whitehouse Canyon Road, Green Valley, AZ, 85614, US

(21) Appl. No.: 11/890,027

(22) Filed: Aug. 2, 2007

Related U.S. Application Data
(60) Provisional application No. 60/835,317, filed on Aug. 2, 2006.

(51) Int. Cl. A61B 18/00

(52) U.S. Cl. 600/439

(57) ABSTRACT

A system is disclosed for the diagnostic or therapeutic treatment of a patient's neural tissues. The system includes at least a means to directly or indirectly acoustically expose or acoustically displace at least a first tissue portion. The system further includes a means to impose, directly or indirectly, an electromagnetic, electrical, magnetic or optical field on at least a second neural tissue portion. The combined or cooperative action of the acoustic exposure/displacement and the field, whether sequentially or simultaneously applied, causes at least one diagnostically or therapeutically useful mechanism. The two tissue portions may be the same portion or different portions.

### CROSS-REFERENCE TO RELATED APPLICATION

The present application claims priority from provisional application Ser. No. 60/835,317, filed Aug. 2, 2006, the contents of which are incorporated by reference herein.

### BACKGROUND ART

### A. Electrical or Electrostimulation:

Electrostimulation of the brain currently mainly involves the invasive insertion of fine electrodes into brain tissues, typically under some sort of image guidance such as MRI or fluoroscopy. The electrodes or needles are frequently held in stereotactic fixtures. Once the electrodes reach their targeted points, typically under stereotactic image guidance, they may be used to monitor or deliver electrical signals. Such work, until recently, has been primarily for diagnostic purposes and the patient typically must have a portion of his/her skull opened. Obviously, then, this diagnostic procedure is used with great reserve and substantial discretion due to its expense, invasiveness and non-zero (e.g., infection) complication risk. A given electrode may have one or more electrical contacts to its surrounding tissues.

Very recently, implanted electrostimulators such as neurostimulators have been introduced to treat ills such as Parkinson's disease, epilepsy, chronic pain and even depression. These devices, some of which are commercially available and approved by the FDA, utilize brain and/or spine implanted or at least invasive electrodes. Such electrodes are typically connected to an electronics module that is itself preferably implanted under the skin like a conventional pacemaker.

In all cases, the above devices involve invasive electrodes whose placement may involve several placement attempts and associated fine-adjustments. In many of these devices, the electrodes are physically placed largely by trial and error. In other cases, multi-contact array-electrodes are provided and the best operating contact is electrically selected for a given array location. Such invasiveness and trial and error means that these types of devices are expensive and are typically used for their diagnostic or therapeutic purposes only when everything else has failed and the patient has suffered from a prolonged period through such failures. However, many refractory diseases have no other treatment options if drugs fail.

### B. Magnetic Stimulation:

Also recently, reports of magnetic stimulation have appeared. In general, magnetic fields of a global or modestly-focused shape are applied non-invasively through the skull upon and/or into the brain, at least into the superficial cortical regions. Note that these fields are applied magnetic fields. It will be appreciated that applied magnetic fields cannot easily be finely focused, particularly at any appreciable depth inside the skull. Thus, these magnetic stimulators are regarded as global or regional diagnostic and/or treatment devices. Their mechanism of operation is not understood but it is thought by some clinicians that neural tissue undergoes some sort of “reprogramming”. Critical here is to recognize that magnets of any type do not have the ability to deliver a fine field focus at depth selectively. Such magnetic fields are inherently global and of sizes many times the size of the magnet itself. Thus they are not suited for selective at-depth neural tissue treatments. My interest herein is to be able to physically address and manipulate neural or spinal tissues on the order of 1 mm in size and locational resolution, possibly situated at some depth.

### C. Other Art:

“In vivo Imaging with Vibro-acoustography” by M. Fatemi et al (Proceedings of the 34^{th} Annual UIA (Ultrasonic Industry Association) Symposium, 2005 (invited)) describes work being done at Mayo Clinic to stiffness-image or spatially map calcifications or tumors in human tissues. It has also more recently been used to stiffness-image hard venous and arterial plaques. A very special type of acoustic transducer is utilized, an overlapping beam dual-beam transducer, to create a tiny mechanically excited tissue region whose responsive motions (or lack thereof indicate the tissue's local hardness. The two acoustic beams overlap in space and create a relatively low frequency mechanical excitation which is at the difference-frequency of the two higher frequency beams. The size of this mechanically vibrated region is on the order of a 1 mm sphere and it can be spatially addressed transcranially as by utilizing trans-skull aberration-corrected beamforming methods known from prior tumor-ablation work utilizing high intensity focused ultrasound or HIFU. All such ultrasonic dual-beam transducers delivering localized vibratory radiation forces, to-date, have been used for such tissue mechanical property imaging. I, however, utilize the acoustic radiation-force induced motions or low frequency vibrations for an entirely unrelated and different purpose, namely, that of creating localized electrostimulation in neural tissues such as by physically moving electrically conductive neural tissues within an imposed enveloping magnetic field. Note the ability to move tissues at a targeted localized tissue portion utilizing the dual-beam transducer technology. The magnetic field does not have to be finely focused, and in fact is best weakly focused and fairly uniform in the region of interest. That is straightforward with magnet technologies-whether permanent magnets or electromagnets. Thus, in order to produce a responsive voltage (or current) pulse in conductive tissues, I utilize a finely focused vibrator acoustic beam overlaid on a relatively uniform imposed magnetic field. Physics dictates that, just like for an electrical generator, a moving conductor in a magnetic field will generate a voltage or current via the known vector cross-product mechanism.

“Resetting the Brain” by C. Gorman (Time, pp. 58-59, Mar. 21, 2005) describes tRMS or repetitive transcranial magnetic stimulation. Essentially, weakly focused pulsed magnetic fields produced by pulsing electromagnetics placed next to the skull have been shown to induce electrical currents in neural circuits and pathways at certain frequencies and to suppress them at other frequencies. Depression, Parkinson's, stroke and epilepsy patients are being clinically tested under the supervision of governmental bodies. Note that the focusing ability, particularly at depth, is quite weak. This means that the treated portions are not at all localized and can be as large as an entire brain quadrant. The induced currents seem to reset the neural circuits in many cases-almost like a reboot of a computer.

“Zapping Away the Blues” by S. Moore (IEEE Spectrum, pp. 16-17, May 2005) describes a “pacemaker” for the brain. The pacemaker-like implanted device sends electrostimulative signals to the left vagus nerve in the neck and has been demonstrated to alleviate symptoms of depression. This beneficial phenomenon was discovered in patients who originally received the implant to treat epilepsy.

“Effects of Low Intensity Ultrasound on the Conductive Property of Neural Tissues” by S.-H. Wang et al (Ultrasonics Symposium, Vol. 3, pp. 1824-1827, 2004) reviews prior acoustic neural stimulation work and demonstrates optimized acoustic parameters for reversibly modifying the CAP or “compound action potential” and/or the CV or “conduction velocity” in targeted neural structures ex vivo.

“The Electrical and Chemical Responses of Neurons” (http://www.uni.edu/walsh/ELECACT.html) outlines the mechanisms which build-up potentials and trigger related currents in neural structures. The movement, pumping and release of sodium and potassium ions are central. Depolarization allows for sending axonal signals and hyperpolarization for inhibiting neuron firing.

“Magnetic Stimulation of Curved Nerves” by A. Rotem et al (IEEE Transactions on Biomedical Engineering, Vol. 53, No. 3, pp. 414-420, March 2006) describes the effects of nerve curvature on their ability to be excited by variable magnetic excitation fields and the electrical fields they produce inside the brain. In particular, the authors study the interaction of induced electrical fields (induced by the electromagnetic stimulator) and the shape of the nerve and its orientation relative to that local electrical field. They relate the phenomenon of stimulation from a passive state to the length constant of the nerve. They also describe diseases that are caused by the length constant being degraded below its normal proportional value relative to the mean internodal distance.

These works demonstrate that magnetic fields, particularly ones whose flux lines move with respect to tissues, can affect the tissues macroscopically. However, none of this work suggests or teaches how one could locally selectively stimulate a specific neural or spinal site because the magnetic-only techniques have very poor focusing ability and those researchers do not teach the herein-invented method of combining high-resolution tissue vibration with low resolution (blanket) magnetic fields to create cross-product induced voltage or current pulses. My technique herein provides the ability to finely probe the brain or spine for diagnostic or therapeutic purposes.

### BRIEF DESCRIPTION OF THE DRAWING

The sole FIGURE is a schematic view, partially in section, of neural tissue stimulation, in accordance with an embodiment.

### DETAILED DESCRIPTION

An apparatus that utilizes at least one acoustic-tissue interaction mechanism for the purpose of contributing to the stimulation, assessment, mapping or treatment of neural tissues is taught herein. A targeted neural tissue portion is exposed to a first fine-focused acoustic beam or energy and the acoustic energy causes an at least temporary change in a tissue parameter of the tissue portion. In supportive combination, either sequentially or simultaneously, a second energy or potential field, typically of a different type, is also applied at least to the tissue portion. The combined or cooperating first and second exposure types acting on the tissue portion, at least one of them preferably finely spatially selective in nature, together cause a change in a neural parameter in, at or near the tissue portion useful to the stimulation, assessment, mapping or treating task. The changed neural parameter could be, for example, the buildup of a neural or axonal potential, the release or relaxation of a neural or axonal potential, the triggering of a neural or axonal potential, the suppression of a neural or axonal potential or triggering event, a change in a neural or axonal time constant, a change in a neural or axonal conductance length, or the creation or triggering of a neural or axonal conductive event or dipole potential. Alternatively, the neural or axonal change could be the therapeutic treatment or surgical destruction of a neural or axonal cellular or signaling apparatus deemed to be problematic or diseased. By “neural” I mean associated with any of the brain, spine, nervous system or nerve structure anywhere in the body.

The acoustic beam or energy, typically selectively or directionally aimed, can even be directed through the skull non-invasively, including with aberration corrections to overcome acoustic defocusing due to the skull. This allows for a fairly fine focal volume to be spatially addressed-on the order of 1 mm in diameter. In a first approach, the acoustic energy causes the targeted tissue portion to oscillate or physically move. In a second approach, the acoustic energy causes the targeted tissue portion to undergo a stationary or pseudo-stationary change in a tissue parameter. In both cases, the acoustically-induced tissue portion change, in cooperation with the help of the separate second applied field or energy, allows the user to cause a selective neural or axonal change at the tissue portion not easily or not non-invasively, possibly with such spatial fineness without the cooperating first acoustic and second energy applications, which may be applied one or both of sequentially or simultaneously.

Note that the change of a neural parameter is the object, whether the change is temporary or permanent. At least the combined or cooperating first and second applied fields or energies will result in a changed neural parameter; however, the first acoustics and the second applied field may or may not individually be capable of causing a neural change. As an example, one or both energies or fields applied alone in isolation may cause a physical change (e.g., temperature change, electrical field change, ion concentration change) but not necessarily a neural parameter change. The key to the invention is that the cooperation of the two applied fields or energies together or synergistically cause a desired particular neural parameter change or changes in a targeted tissue portion(s).

The first acoustic application means and the second energy or field application means may one or both be non-invasive, minimally invasive or invasive in nature. The invention may be utilized in a doctor's office, clinic or hospital, for example, for screening, diagnosing or beneficially therapeutically or surgically treating human or mammal subjects. A robotic system may also be utilized to deliver the inventive diagnostic or therapeutic treatments. In some cases, an ambulatory or out-patient therapy device is provided by a carried, portable or implanted device.

The acoustic beam or energy is typically delivered using an acoustic piezo-transducer, however within the inventive scope is the use of other acoustics-production mechanisms such as thermoacoustics, magnetoacoustics and photo-acoustics to produce the needed acoustical energy remotely or directly at or in the tissue portion. A contrast agent may be used with the invention to enhance its performance in several manners.

A preferred acoustic transducer is a dual beam transducer, such as a dual beam annular array transducer, preferably wherein an inner annular array and an outer annular array operate at somewhat different frequencies, say each in the megahertz (MHz) range. The two beams overlap and are preferably concentric. In this manner, at the beam overlap focal region of the dual transducer, I have not only the two mentioned high frequencies present but also have the expected sum and difference frequencies of those present. It is preferably the low difference frequency that I utilize. Note that the difference frequency can be arranged to be anything I want, and kilohertz (KHz) range difference frequencies whose timing (risetime, period, etc.) are similar to that of actual known neural pulses are highly useful to stimulate neurons and axons. These low KHz or lower range frequencies are able to microvibrate (or singly displace on a one-pulse basis) the targeted tissues as they do for known acoustovibrography (stiffness mapping) techniques.

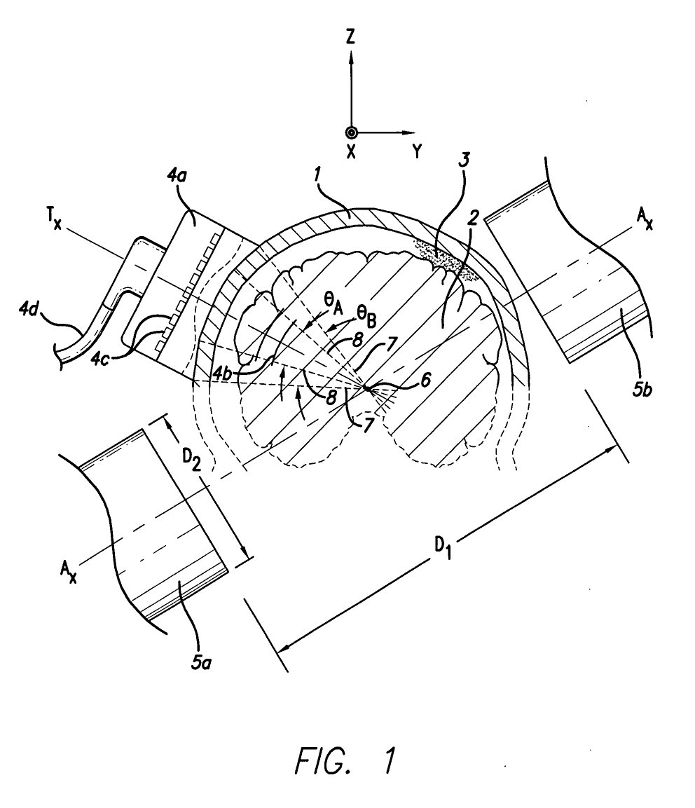

The invention is depicted in attached FIG. 1. Therein a patient's skull 1 is shown in sectional view. The skull bone 1, cerebrospinal fluid 3 (CSF) and brain matter 2 can be seen. A reference coordinate system with X, Y, and Z axes is depicted at the top of the drawing. For simplicity, I have not shown dura matter and blood under the skull bone.

An ultrasonic or vibratory acoustical-energy transducer 4a is depicted acoustically or mechanically coupled to the patient's skull 1. Typically, this transducer may be a focused mechanical or focused phased-array device. Thus, the ultrasonic transducer 4a will also typically include piezoelements 4c and a power cable 4d. Note that I depict a couple of overlapping acoustic beams defined by phantom lines 7 and 8. Beam 7 is an outer beam and is depicted as having an included angle of Θ_{B} and beam 8 is depicted as having an included angle of Θ_{A}. Typically, outer transducer annular ring electrodes will be dedicated to the outer beam 7 and inner transducer ring electrodes dedicated to the inner beam 8. Note that in this arrangement the beams primarily only overlap at a common focus or focal region 6 where I obtain the desired difference frequency. More details about the acoustic beam geometries and frequencies are provided in the Fatemi et al article, supra. Note, however, that transducer 4a coaxially applies its emitting acoustic energy at the two slightly different frequencies, generally along the Tₓ or transmission axis at the moment depicted. This is depicted as ingoing acoustic waves 4b.

Next, in FIG. 1, two depicted electromagnets or permanent magnets 5a and 5b are shown aligned along the axis labeled Aₓ. Assuming for the moment that these are electromagnets, it will be seen that they have a face diameter of D₂ and a relative separation distance from each other of D₁. It is known that one can apply static or variable transient (e.g., pulsed) magnetic fields using one or more (two shown) such electromagnets or permanent magnets. The magnetic fields extend into the patient's skull 1 and brain 2 as expected for a magnet pair. Permanent magnets would be limited to static or pseudo-static fields unless they were mechanically scanned or moved relative to the skull. Within my inventive scope is any imposition of magnetic fields upon my region(s) of interest. Such field strength lines may be static (with respect to the overall skull), dynamic (in strength or shape) or purposely physically scanned. In all cases, the magnetic field, assuming it is applied non-invasively to subdural tissues, will not be finely focused as my acoustic transducer can be, unless it is applied by a very small or needle-shaped magnet. Also included in the scope of the invention is the invasive application of the magnetic field(s) such as by an invasive permanent magnet, electromagnet or superconducting magnet. I find that MRI systems provide a convenient source of strong fields. Preferably, the magnetic field is static but it may also be pulsed or CW. Preferably, the magnetic field has some gradient to it to induce more current in the moving conductive neural tissue. The invention is not limited to use of dual magnets. More magnets or single magnets may alternatively be utilized.

Given the physical arrangement of the invention, it should be apparent that one can impose upon a tissue portion both a static/variable magnetic field that is weakly focused if not relatively locally uniform (on the scale of my 1 mm or so target regions) and an acoustic radiation-forced displacement due to the transducer. Keep in mind that by locally uniform I can mean that a desired and preferred magnetic gradient is locally uniform. This may be done either of sequentially or simultaneously. In any case, wherein the neural tissue (e.g., an axon) has a conductivity or current, whether sub-threshold and passive or super-threshold and active, one can introduce vector cross-product interactions. Examples of this are as follows:
1) A potentially conductive or activatable neural path movable by acoustic displacement in the applied magnetic field will experience an applied transient potential in accordance with the movement or displacement waveform. The acoustical energy by itself, the magnetic field by itself, or a delivered conductive contrast agent may any or all of serve to induce conductivity or activation. The combined action of the acoustic energy and the magnetic field may also induce conductivity change. These may also cause the neural tissue to be raised over a conductive threshold in combination with natural neural processes and potentials.
2) A presently conducting or activated neural path moved by acoustic displacement in the applied magnetic field will likewise receive a transient applied potential. This potential may be additive or subtractive to a natural current or potential. Likewise, the inherent momentary conductivity of the neural tissue may be modulated as in (1).
3) A sub-threshold neural path may be sent super-threshold by either or both of an induced transient potential via displacement in the magnetic field or via the application of any of the acoustic energy itself, the magnetic field itself or by the combined acoustic and magnetic energy and field.
4) A super-threshold or super-threshold-capable neural path may be rendered sub-threshold as for (3).
5) Before applying both magnetic field and acoustic displacement field for their combined effect(s), one of the magnetic field or acoustic displacement/exposure may be preapplied to modify any of i) one or more CAPs or compound action potentials, ii) one or more CVs or conduction velocities, iii) one or more length constants, iv) one or more internodal distances, v) one or more neural or axonal orientations relative to each other or relative to an applied magnetic field or displacement field, vi) the curvature of a neural or axonal structure, vii) a tissue temperature, viii) a cellular membrane permeability, and/or ix) the concentration of a delivered or targeted drug or contrast agent.
6) Same as (5) but while still imposing (5) the other driver (magnetic field or acoustic radiation is also applied to get the combined effect. In this approach, (5) early on acts to precondition the neural structures. Post-conditioning is also possible when an additional follow-on cycle is planned.
7) Measurement or mapping of conduction length constants, threshold values, connectivity, degree-of-isolation.
8) One or more neural parameters is substantially reversibly manipulated, regardless of whether the manipulation involves hysteresis or not (see Wang et al reference, supra).
9) Rearrangement of or creation of neural connections or signaling pathways.

Therefore, I can manipulate sub-threshold passive properties below their threshold values or to and above their threshold firing, saturation or triggering level. Further, I can interfere with super-threshold states by suppressing action potentials. I can also amplify preexisting sub-threshold or super-threshold signals. The effect of the acoustic exposure itself can modify the status of the axon ion-pumps and the sheath potentials or the axon membrane potential. It is widely known that membrane permeability is enhanced with ultrasonic exposure. The stimulation time-constant and the membrane time-constant will usually be manipulated in a controlled relationship to each other to observe thresholds.

Because I preferably, but not exclusively, may utilize an acoustic transducer having overlapped beams, I can set the sum and difference frequencies of the two beams at the overlap region to whatever I wish, say 10 KHz, for example. Most commonly, I may set the difference frequency to a value having a known relationship to (e.g., equal to, above or below) a neural time constant. Thus, the overlap excitation will be highly focused and have a cyclic or single-pulse waveform, depending on how the two transducer portions are driven. In this manner, I excite neural entities at or near their natural excitation frequencies or time constants.

One would likely choose the two transducer frequencies to be close to each other (e.g., within, say, a few KHz to 10 KHz excitation difference frequency) yet both still at or below 2 MHz. This will minimize attenuation in the skull. For example, the two transducer frequencies may be 2.000 MHz and 1.990 MHz, giving a delta frequency of 0.010 MHz or 10 KHz in the common focal region. Because of the high (2 MHz) frequencies, the focus is highly resolved yet I get a low frequency radiation-force tissue motion at that finely resolved focus.

One may also choose the two frequencies to be higher but may have to provide a cooled contact or standoff to prevent unacceptable skull heating for any significant transducer operational duty cycle. Skull losses go up appreciably above 2 MHz. I include in my inventive scope the delivery of a catheter transducer into the patient's skull, for example. Such a catheter transducer can serve to receive or transmit pinging signals useful to determine whether aberration corrections are working. I note that although I have mentioned annular concentric arrays, the ideal transducer would be coaxial beams coming from a two-dimensional (2-D) matrix array transducer. Using such a 2-D array transducer allows for a full 2-D aberration correction to be accomplished, whereas an annular ring transducer can only apply an approximate radial correction alone.

Readers of the Fatemi et al reference will realize that the overlapping beam approach allows for one to achieve a very large displacement at a very small volume location. This is not easily done with a single frequency non-overlapping beam system. In addition to the displacement, one also enables low frequency (KHz, for example) palpation or pressurization/rarefaction of the tissue portion with a period comparable to neural time constants. Within my inventive scope is the use of single beam and single frequency transducers as well, including use of just one of the dual beam transducers at a time to manipulate or treat the tissue portion or image the tissue portion. One might also take measures to somewhat defocus the transducer. One might also take measures to somewhat defocus the transducer to mechanically excite a larger volume of tissue.

Note that at the overlapping beam common focus I have, from the above example, difference and sum frequency acoustic exposures of both 10 KHz and 3.990 MHz. The action of the low frequency tends to be displacement and possibly cavitation if the power is high enough. The action of the higher frequency tends to be heating (depending on power level) and changes to membrane permeability and ionic diffusion. Further, the high frequency in particular can cause some microstreaming of CSF or blood (or a drug or contrast agent therein) at the tissue portion along with the tissue translation or displacement. In fact, much of the sum frequency will be attenuated in the skull (for the non-invasive neural application) such that neural tissue targets will not be unacceptably heat unless that is desired. I can easily cool the skull-applied transducer and its underlying skullbone with a liquid filled or liquid flowing standoff if that is necessary.

Since one can steer the overlap focus point or region mechanically and/or electronically (depending on transducer arrangement), then one can selectively excite any desired portion of the neural matter. Since I preferably utilize the dual transducer (mixing beams in the most general description) to mechanically excite the selected local tissue portions with, preferably, the difference frequency, I can also image the induced tissue displacements by using one or both transducer portions as an acoustic imaging means. Note that utilizing a phased array transducer, one could excite multiple neural targets simultaneously in very close sequence. This may be very useful for creating an action potential and following its propagation.

I emphasize that the invention can also work using a single beam transducer, but for the highest resolution higher frequency beam-mixing is preferred. Further, given the radiation-force idea taught here for electroprobing, I also notes that radiosurgical treatment equipment will also apply a similar radiation force as due to the impacting particle or energy beams. Such cancer-treating radiosurgical beams can also be applied with very high spatial resolution.

Within the scope of the invention is any part of the displacement-inducing transducer also being capable of doing acoustic imaging or acoustic measurements of tissue thicknesses, velocities or displacements. Likewise, the transducer(s) may be utilized to necrose dysfunctional or diseased tissues or may be used to deliver a drug as by a cell membrane permeabilization mechanism or contrast-agent bursting mechanisms.

Within the scope of the invention is any applied transducer acoustic signal, whether a frequency sum, difference or neither, having at the focus a pulse period below, at or above an inertial (motion related) time constant of the targeted tissue or portion thereof.

Within the scope of the invention is any applied transducer acoustic signal, whether a frequency sum, difference or neither, having at the focus a pulse period or pressure bearing a relationship to a neural time constant, a neural length constant or a neural ionic diffusion time-constant.

Within the scope of the invention is the use of any acoustic transducer which can at least one of a) acoustically image, b) displace tissue, c) excite tissue due to its applied pressure or rarefaction, d) stun or kill tissue, e) reversibly or non-reversibly change a neural parameter of at least one tissue portion whether helped by an applied or induced field or not, e) heat and necrose or ablate tissue, and/or f) beneficially manipulate a neural signaling pathway.

I emphasize that the imposed tissue motion may be of a single-pulse, multipulse or pseudo-static nature. It is the tissue motion waveform within the imposed magnetic field that determines the induced potential waveform. However, it is known, as mentioned, that any acoustic or magnetic exposure can alter tissue electrochemical and physical properties-focused acoustics on a highly resolved basis (if desired) and magnetic fields on a coarser more-global gradient basis. Thus, I anticipate a toolkit wherein tissues can be globally or locally pre-conditioned (or post-conditioned) before (or after) highly localized application of my preferably non-invasive electroprobing method. I also anticipate the inventive equipment being physically manipulated such that the desired vector cross-product of magnetic and displacement fields is maximized in magnitude, such as orthogonal, for example.

It is anticipated that the invention herein may be utilized together with other imaging tools such as functional MRI and/or PET and/or infrared subdural imaging such that additional information can be gleaned from the patient's procedure.

I have discussed using the depicted dual magnetic fields as the second applied energy or field. Such magnets will typically be pulsed or cyclically operated electromagnets, but I also include in the scope of the invention static permanent or unpulsed electromagnets. Even with these, one can excite axons by acoustically moving neuronal tissue in their typically somewhat non-uniform fields. The system may utilize one, two or more magnets. In an MRI system, the gradient magnet functionality may be utilized to support the invention.

Any of the magnets or acoustic sources may be non-invasive, minimally invasive, or completely invasive. Assuming non-invasive acoustics, I preferably apply known beam aberration corrections to the trans-skull delivered acoustic beam to compensate for the skull thickness and shape variations. The acoustics (or second fields) may alternatively be applied by a catheter or scope-mounted device. Included in the inventive scope is the use of the MRI (or a different MRI session or machine) to compute these aberration corrections, at least approximate ones, in the known manner.

I anticipate the apparatus may be utilized for diagnostics, mapping and for therapy, including schemes wherein both diagnosis and therapy takes place in the same session or at least with the same inventive apparatus. By “therapy” I mean any exposure(s) that leave the neural tissues and patient in an improved condition after some extent of treatment(s) or exposure. Note that therapeutic treatment can include purposely damaging (or not) selected neuronal structures as by utilizing controlled induced currents or by delivering high-intensity necrosing ultrasound heating therapy. rtMS has already demonstrated a resetting of neuronal structures apparently without significant permanent damage done to the brain matter.

Included in the scope of the invention is the application of static or dynamic electric fields rather than or in addition to magnetic fields; however, it is thought that for non-invasive work, it is preferred and somewhat safer to utilize magnetic fields due to the shock hazard of the required electrical fields. A possible alleviation of this concern could be provided by using an EMP or electromagnetic pulse rather than a static or pseudo-static field. Such pulses can be delivered from, for example, capacitor or plasma discharge devices.

So, I can generate, suppress or even amplify neural electrical pulses (voltage or current) at brain target portions such as 6 by imposing a magnetic field from magnets such as 5a and/or 5b and vibrating or pulse-moving the conductive or potentially-conductive tissue in that field at the tissue-portion target portion, using the acoustic or vibratory means 4a. Thus, I have a moving conductor (brain ionic conductive pathways) in a magnetic field and therefore I create an electrical pulse in accordance with the acoustic physical displacement pulse waveform, assuming a static or gradient magnetic field. It is to be noted that tissues have natural passive operational conductivities at various times of their passive subthreshold functioning and that the application of the acoustics and/or magnetics can also create, amplify and/or release such currents.

A major innovation of this invention is that in order to make it work on selectively finely-targeted regions such as 6, one must be able to deliver at least one of the acoustic or magnetic field spatially-selectively with the desired temporal control. I do this for electrostimulation herein in a manner similar to that done for the emerging field of acoustic elastography or acoustovibrography (Fatemi et al reference). In that field, one selectively vibrates or displaces tissue portions and estimates their mechanical properties from their resulting motion(s). That prior application is purely mechanical and related to mechanical tissue properties only. However, herein, I use that vibration or tissue-distortion approach to vibrate or displace my target tissues selectively within a larger applied magnetic-field region to induce or otherwise interfere with neural electrical currents and/or potentials.

Those familiar with ultrasound and the skull 1 will be aware that the skull 1 presents a significant impediment to the passage of ultrasound and to the focusing of ultrasound.

The first impediment is acoustic attenuation, particularly above about 2 MHz. However, my application herein requires, in many of its forms, only a small or low duty-cycle, meaning that I can tolerate significant acoustic attenuation because significant interleaved heat-sinking can take place such as into the cranial blood supply or into an external cooling member.

The second impediment is that of acoustic aberration wherein an acoustic wavefront, such as that depicted by 4b, non-uniformly progresses through the skull. This results in an unintended defocusing effect. This is mainly because of the variable thickness of the skull as depicted in FIG. 1. However, recent progress in delivering surgical ultrasound or HIFU into the brain through the skull has taken advantage of aberration correction methods wherein appropriate phase-delay corrections are applied to each emitting piezoelement. In this manner, it has been proven that a fine acoustic focus can still be obtained in brain matter despite the beam having passed through variable thickness skull regions, provided such aberration correction is utilized. If I utilize an MRI system as my source of magnetic fields, I can conveniently also utilize it to estimate the aberration corrections based on images of the variable-thickness skull.

Included in the scope of the invention is the delivery of acoustic radiation force from any access path, including through the skull, through the nasal passages, through the eyeballs and/or sockets and through the temples.

I have said that I vibrate or displace the targeted tissue portions such as 6, preferably with a displacement waveform related to a desired produced electrostimulation waveform. The challenge is to do this with preferably significant amplitude (1-100 microns, for example) on a highly localized basis. The overlapping beam method of elastography or acoustovibrography is quite capable of this. Such motions of conductive paths in the applied second field cause appreciable stimulated currents and potentials. I note that by displace I mean at least in one direction for at least a period. Thus, a displacement may be “held” in place for a time and the transitions examined for their induced current excitations.

It is anticipated that the invention will be utilized in combination with several existing diagnostic and research techniques. Multimodal functional neuroimaging (e.g., fMRI) has received significant attention in the past few years and has already advanced the understanding of the spatiotemporal pattern of brain activation and connectivity underlying perception, motion and cognition. My invention herein extends that imaging and probing data-gathering ability to even higher resolutions as well as provides a tool for manipulating (therapeutically or surgically) the tissues as well or instead. The existing neural mapping tools include the non-invasive brain imaging techniques of fMRI or functional magnetic resonance imaging, high resolution EEG or electroencephalography, event-related brain potentials (ERPs) and magnetoencephalography (MEG), positron emission tomography (PET), and single positron emission computed tomography (SPECT). MEG has enabled the estimation of the neural sources from the scalp potentials or magnetic fields recorded through high-density sensors by solving the so-called inverse problem. However, due to the ill-posedness of the inverse problem, these techniques suffer from the ambiguities in defining the precise locations of brain activity. Of the prior art techniques, only fMRI has reasonably high spatial resolution; thus, my inventive technique can offer spatial information beyond what these other prior techniques offer (or more inexpensively than they offer) and can be employed to map neurology by itself or in combination with these prior techniques. The prior techniques such as EEG and MEG allow the mapping or location of cortical electrical currents on a rather coarse basis. My invention, because it can excite (or suppress) tissue currents on a very localized basis, will allow improved mapping of functionality and electrical states vs. EEG and MEG used alone or instead. Combined prior art mapping might utilize surface, cranial-screw EEG or sub-dermal ECG electrodes, for example. In any event, this invention will allow a higher-resolution surgical planning to take place than previously.. The therapy or surgery is also at a higher resolution and is at least less invasive.

Several afflictions, such as stroke, affect synaptic transmission, axonal conduction, and cellular action, exhibit potential firing in a sequential manner. The invention should allow both a more spatially detailed view of the damage from stroke but also the possibility of treating the damaged areas selectively and even non-invasively. The invention is seen as the first tool wherein one can probe and/or correct neural function on a localized neural basis.

Highly resolved information on cortical connections and connectivity can be gathered using the teachings of the invention. This could be for diagnostic, therapeutic or surgical reasons. Intraoperative neurological monitoring, the evaluation of the neural system during surgery, is also expected to be an application. This is particularly true for cases wherein the invention is used to determine connectivity or functionality, if not also to operate in a necrosing or tissue-altering mode to destroy, create or modify neural connections. Recall that therapy can also be provided at sub-necrosing energy levels, as it is widely known that cell membranes and axonal membranes can be diffusionally manipulated even with low-intensity ultrasound. The sources of INM signals may, for example, be muscle activity (EG), heart activity (ECG), or eye-movement artifacts (EOG). Information on the location of current-dipoles in the brain can also be produced.

A further new neural imaging and neural measurement technique is that of functional near-infrared spectroscopy of fNIR. An advantage of this technique is that it can map cortical regions non-invasively in a doctor's office. It can also be used in combination with the invention herein, for example to refine fNIR resolution further. 1 add that the combined or cooperating second applied field or energy and the first applied acoustic displacement (or acoustic energy exposure) do not necessarily have to occur simultaneously, although that will be a common choice to get the vector cross-product effect. For example, a preapplied field or energy may be used to precondition the tissue, globally or locally, such that the localized displacement better induces a local voltage or current. In this manner, the applied second field and the first applied displacement (or acoustic energy exposure) are supportive or synergistic of each other but not necessarily simultaneously applied. In such circumstances wherein the second applied field and the first applied displacement (or acoustic energy exposure) are sequential or interleaved, there will likely be a potential, polarization, dipole or conductivity-relaxation time-constant within which the follow-up second mechanism (field or displacement) must be applied in order to get the cooperative effect. Such time constants are known in biomedicine and diffusion/relaxation phenomenon throughout physics.

Note that in my use of “first” and “second”, I do not imply any temporal sequence or simultaneity. I mean only to differentiate between the first applied acoustic energy, field or beam and the second, probably differently sourced, applied electromagnetic field, beam or potential. Note also that I specifically mentioned the second applied field as being electromagnetic or magnetic in nature, or even comprising an electrical steady or transient field. The second field might also be an optical field as for the mentioned fNIR field, for example, or for steady or pulsed blanket, regional or targeted NIR. I taught that the first and second applications may be applied in any order, sequence or manner of simultaneity or overlap.

Also included in the scope of invention is the use of the invention to propagate or pump signals through multiple neuronal connections from region to region. This can be done by sequentially or simultaneously applying my inventive stimulations or exposures along such an intended path in the brain or spine. This allows the user to selectively activate (or create) neuronal signal paths requiring multiple stimulation and/or monitoring points or changing points. Such exercises are quite impractical invasively due to the large number of electrode manipulations required.

Assuming that one can propagate signals over macroscopic distances in the brain by manipulating multiple tissue sites in temporal cooperation, it would be possible to mimic the natural operation of such signal pathways. It may even be possible to program learning or behavior benefiting the patient. An application of such a capability might usefully be to treat a treatment-resistant or rehab-resistant addiction or to treat the brain-damage resulting from a severe stroke. In such a circumstance, medical ethicists should be consulted to assure the motivation is properly to help or benefit patients. That is the sole intent herein.

A further interesting possibility is that non-natural neural pathways might be formed or implemented. By “non-natural” I simply mean they were artificially created, as opposed to naturally generated. Such pathways and connected cellular apparatus may be useful to cure a host of neural diseases and possibly treat addictions, depression, motor-diseases and other maladies such as obesity. Such artificially produced pathways may replace dysfunctional or missing ones, offer redundancy, or introduce new cognitive functionality. It may be the case that the brain's impressive plasticity allows for the invention to overlay corrective or added functionality without harming any existing functionality.

I mention that tissue “displacement” using the acoustical energy includes tissue distortion, the result still being that at least some tissue has moved.

What is claimed is:

1. A system for the diagnostic or therapeutic study or treatment of a patient's neural tissues which includes at least:
a means to directly or indirectly acoustically expose or acoustically displace or deform at least a first tissue portion;
a means to impose, directly or indirectly, an electromagnetic, electrical, magnetic or optical field on at least a second neural tissue portion;
said combined or cooperative action of said acoustic exposure/displacement and said field, whether sequentially or simultaneously applied, causing at least one diagnostically or therapeutically useful neural activation, switching, suppression or modulation mechanism; and
the two tissue portions being the same portion or different portions.

2. The system of claim 1 wherein said displacement or deformation is displacement or deformation of a neural tissue or cellular entity or entity-portion capable of being electrically or electrochemically activated, switched, suppressed or modulated, said displacement or deformation taking place in said field, said displacement or deformation in said field causing a displacement-field or deformation-field interaction contributing to the neural activation, switching, suppression or modulation.

3. The system of claim 2 wherein the imposed field is a magnetic or electromagnetic field preferably including a field gradient versus distance or versus time at the tissue portion.

4. The system of claim 1 wherein said diagnostically or therapeutically useful mechanism is at least one of:
a) causing a neural structure to aftain or retain a super-threshold state;
b) causing a neural structure to attain or retain a sub-threshold state;
c) causing a neural structure to have a modified CAP, CV, membrane potential or membrane time constant;
d) causing a threshold state to be identified or quantified;
e) causing an axonal current, axonal potential, or neural triggering event or state-modification of any type;
f) causing a neural structure to be deformed such that it interacts or interacts differently with an applied field or has a changed neural parameter;
g) causing a change in a neural length constant;
h) causing a change in a neural internodal distance;
i) causing a change in an ion concentration or diffusivity in or around a neuronal structure or axon;
j) changing a neural threshold level or suppressing a neural threshold;
k) mapping any neural parameter with two or more data points;
l) changing, transiently or permanently, a conductivity or electrical property of a neural path; or m) modifying a myelin sheath of an axon in any manner causing a neural parameter to change.

5. The system of claim 1 wherein the one or two tissue portions are any of:
a) are the same portion or overlapping portions;
b) have or should have a known signal path between them, whether functional or not;
c) may have a signal path between them, whether functional or not;
d) may have a new signal path created between them;
e) may have a preexisting signal path between them modified or destroyed transiently or permanently;
f) may have a stimulated signal passed between them;
g) may one or both be triggered;
h) may one or both be set or limited to a sub-threshold value;
i) may one or both be set or limited to a super-threshold value;
j) may one or both have its polarization or charge state manipulated;
k) may one or both be killed, necrosed or disabled;
l) are in a cortical or brain-stem region;
m) are treated non-invasively;
n) are diagnosed or mapped and then treated using any treatment means;
o) are diagnosed or mapped and then treated using the system;
p) undergo learning by direct neural manipulation;
q) undergo a treatment which addresses a disease or addiction;
r) are enabled to serve a motor control function, voluntary or involuntary; or
s) become or remain active neural entities after said treatment.

6. The system of claim 1 wherein two or more tissue portions are treated, whether simultaneously or sequentially.

7. The system of claim 1 wherein an electromagnet, capacitive-discharge, EMP or illumination-means is utilized to apply a field, whether operated or applied in a static, steady-state, CW or pulsed manner.

8. The system of claim 1 wherein a permanent or pseudo-permanent magnet is utilized in any manner to apply a field, whether said magnet is physically scanned or not.

9. The system of claim 1 wherein at least one acoustic transducer is utilized to apply acoustic energy.

10. The system of claim 9 wherein at least one transducer is any one or more of: a) a mechanically focused single focus transducer, b) a mechanically focused multifocus transducer, c) a mechanically steerable transducer of any type, d) a phased array electronically steerable transducer of any type, e) one or more transducers emitting or capable of emitting two or more different beams, f) one or more transducers emitting or capable of emitting overlapping, confocal, concentric or mixing beams, g) one or more transducers emitting or capable of emitting at two or more different frequencies, h) one or more transducers whose emissions provide a mixed frequency selected from the group of a difference frequency or a sum frequency, i) an annular array, j) a 2-D array, k) a transducer which produces acoustic radiation forces which displace tissue, or l) a transducer that displaces tissue with a frequency or time constant on the order of a neural time constant.

11. The system of claim 1 wherein at least one 2-D array or annular acoustic transducer or transducers has overlapping, combining or mixing beams, beam-portions or overlapping beam capability and an overlap, mixing or combining region operates with a sum or difference frequency.

12. The system of claim 1 wherein tissue motion, displacement, deformation or pressure is pulsed or cyclic.

13. The system of claim 1 wherein tissue motion or pressure is or can be measured in microns or millimeters in magnitude or in dynes/cm², Pascals, PSI or Bars.

14. The system of claim 1 wherein at least one acoustic transducer causes radiation-force induced motion of or pressure upon a tissue portion capable of at least temporary displacement or deformation.

15. The system of claim 14 wherein said motion or pressure is driven at any one or more of a transducer(s) difference frequency, sum frequency, primary frequency or harmonic frequency.

16. The system of claim 14 wherein tissue motion or pressure is pulsed or cyclic.

17. The system of claim 14 wherein tissue motion or pressure is or can be measured in microns or millimeters in magnitude or in dynes/cm², Pascals, PSI or Bars.

18. The system of claim 1 wherein the system's field and acoustics applicators either are both non-invasive in nature, one is non-invasive and the other is invasive in nature, or neither are non-invasive in nature.

19. The system of claim 1 wherein at least one of an applied field or an acoustic energy is pulsed, varied, switched, transitioned or gated with a time constant or frequency bearing a relationship to a neural time constant or a physical tissue-displacement or motion inertial time constant.

20. The system of claim 1 wherein simultaneous or sequential exposure to the applied field and the acoustical energy causes a cooperative effect in changing a neural parameter transiently or permanently, said cooperative effect not necessarily involving a cross-product interaction between the field and the acoustic energy, said effect still possibly allowing for such a cross-product effect of field and induced displacement before or after.

21. The system of claim 1 wherein any portion of the system is invasive, minimally invasive, or implanted for any period.

22. The system of claim 1 wherein an applied or induced magnetic or electric field vector and an applied acoustic displacement vector or acoustic wave direction bear a controlled, estimated or known geometrical spatial relationship.

23. The system of claim 1 wherein a tissue portion, in at least one direction, has a characteristic dimension on the order of a few millimeters or less.

24. The system of claim 1 wherein an applied or induced magnetic or electric field or an applied acoustically-induced displacement or pressure is scanned through at least one dimension, direction, plane, tissue region, tissue volume, brain portion, spine portion or angle.

25. The system of claim 1 wherein the system, for at least one tissue portion, operates sequentially or simultaneously in diagnostic and therapeutic modes.

26. The system of claim 1 wherein the system excites, triggers or suppresses one or more neural structures, directly or indirectly.

27. The system of claim 1 wherein the system excites, triggers or suppresses an action of a first neural structure and observes an effect at a second neural structure or structure portion.

28. The system of claim 1 wherein a patient provides anatomical or verbal feedback in response to the operation of any portion of the system.

29. The system of claim 1 wherein a drug or contrast agent, targeted or not, is employed to favorably cooperate with the use of the system or to enhance its effect on at least one tissue portion.

30. The system of claim 29 wherein said drug or contrast agent is any one or more of: a) a modifier of a neural parameter, b) a modifier of a neural conductivity, electroconductivity, ionic state or activation state, c) a drug-bearing material, d) a drug-bearing material for acoustic release at a neural site needing drug treatment, e) a biologically targeted contrast agent, f) a material which is acoustically activated in support of a therapy or diagnostic procedure, g) a material that causes or supports a neural parameter change with the help of one or both of the acoustic energy or applied field, h) a material that is used to inactivate, stun, kill or necrose a neural entity, or i) a material that is used, at least in part, to enhance an image or highlight a neural functionality.

31. The system of claim 1 wherein an MRI, fMRI, magnetic, electromagnetic or optical neural mapping, therapy or surgery system independent of the inventive system is utilized in cooperative or supporting sequence with or in parallel with the inventive system.

32. The system of claim 1 wherein a neural threshold value is changed temporarily, transiently, with a frequency, semi-permanently, or permanently.

33. The system of claim 1 wherein a neural threshold state is eliminated or created.

34. The system of claim 1 wherein a neural threshold state is added where there was not a functional one or was not any before it.

35. The system of claim 1 wherein a drug or contrast agent, targeted or not, is employed to favorably modify an ionic concentration, ionic diffusivity, neural time-constant, neural conduction distance or tissue conductivity.

36. The system of claim 35 wherein said modification is targeted to one or more neural tissue portions, with or without the help of said acoustical energy or said applied field.

37. The system of claim 1 wherein multiple points on one or more targeted tissue portions are spatially or temporally addressed so that information pertaining to the connective relationship, if any, between the two or more points can be acquired or deduced.

38. The system of claim 1 wherein any one or more of an applied or induced field or an applied acoustic displacement or exposure causes a triggering or activation of a neural structure approximately at a natural or normal threshold value.

39. The system of claim 38 wherein said triggering or activation is at a non-natural value.

40. The system of claim 1 wherein an applied magnetic, electric or optical field is provided by any portion of an MRI, fMRI, PET SPECT, CATSCAN, fNIR or other imaging equipment, including optical and infrared imaging or spectroscopic scanners.

41. The system of claim 1 wherein an applied electrical field, or at least a momentary electric field vector, is provided by a DC or RF excitation sourced from an fMRI, MRI, PET, SPECT, CATSCAN or fNIR imaging scanner, said electric field being supportive of the diagnostic or therapeutic procedure.

42. The system of claim 1 wherein the patient is imaged in any manner by any other tool in support of the application or use of the system, whether said imaging occurs before, during or after the use of the inventive system.

43. The system of claim 1 wherein the patient has one or more neural structures temporarily, transiently, permanently or semipermanently physically reshaped, modified, created, destroyed or deformed thereby beneficially affecting a neural function of the patient.

44. The system of claim 1 wherein a myelin-related disease is treated.

45. The system of claim 1 wherein a degenerative brain or spinal disease is treated.

46. The system of claim 1 wherein depression, an addiction, chronic pain, obesity, psychosis, epilepsy, stroke-damage or impairment of any type, mental or physical, is treated.

47. The system of claim 1 wherein an acoustic transducer utilizes aberration correction to maintain or attain a fine focus at a tissue site.

48. The system of claim 1 wherein an acoustic transducer or acoustic energy therefrom is one of mechanically steered or electronically steered to at least one subdural tissue site of interest from an invasive or non-invasive location.

49. The system of claim 1 wherein an acoustic transducer or other cooperating imaging means detects or measures an acoustically induced tissue-portion motion or displacement.

50. The system of claim 1 wherein the system as a whole or the acoustic transducer itself stuns, kills or necroses a neural tissue structure in any manner, including via thermal necrosis, spatially selective drug release, acoustic cavitation-assisted death or sonoporation assisted death.

51. The system of claim 1 wherein some mapping of at least one neural parameter is carried out for screening, connectivity-assessment, diagnostic or therapy purposes.

52. The system of claim 1 wherein at least one of the applied or induced field or the applied acoustic displacement or exposure at a tissue portion is single-pulsed or multicyclic in nature.

53. The system of claim 1 wherein an rTMS system or electroconvulsive system is replaced or supplemented by the inventive system.

54. The system of claim 1 wherein invasive brain or spine electrode(s) are replaced or supplemented by the inventive system.

55. The system of claim 1 wherein the patient or treatment subject provides feedback to the system or practitioner to modify the system behavior in any voluntary or involuntary manner.

56. The system of claim 1 wherein at least some data collected by the system is communicated over a communication, instrument or data network of any sort, whether wired or wireless.

57. The system of claim 1 wherein any portion of the system is mounted on or in an invasive catheter, scope, port, surgical tool or implant.

58. The system of claim 1 wherein the patient or treatment subject undergoes one or more diagnostic and/or therapeutic sessions.

59. The system of claim 1 wherein the system is mobile or ambulatory in nature.

60. The system of claim 1 wherein the system is powered by any one or more of battery, fuel-cell, human-energy or electrical.

61. The system of claim 1 wherein the system is provided as a modification of an fMRI, MRI, open-arm MRI, PET, SPECT, CATSCAN or fNIR imaging scanner or of an acoustic imaging or Doppler system.

62. The system of claim 1 wherein the applied field includes both a static or steady-state field and a transient field, said fields being sequentially or simultaneously applied, said fields possibly applying and/or inducing different field vectors to a tissue portion of interest.

63. The system of claim 1 wherein the applied field includes a field gradient or can be modulated or modified to produce a gradient.

64. The system of claim 1 wherein its application is, at least in part, to deduce the particular functional state and/or spatial organization of a particular patient's brain.

65. The system of claim 1 wherein the acoustic energy any one or more of: a) displaces or deforms tissue, b) changes or modulates a neural parameter, c) heats tissue, d) changes a physical tissue parameter such as temperature, or e) releases or activates a drug or medicament.

66. The system of claim 1 wherein an acoustic transducer emits one beam at a time with respect to a given addressed tissue portion.

67. The system of claim 1 wherein an acoustic transducer or transducers emit(s) two or more beams at a time or in sequence along one or more acoustic emission paths or directions, said beams optionally simultaneously addressing different tissue portions of interest.

68. The system of claim 1 wherein a treatment applicator contains or includes both a field applicator and an acoustic transducer.

69. The system of claim 1 wherein the patient or treatment subject wears, lays upon, straps upon him/herself or is otherwise juxtaposed to at least one component of the system for a period of time.

70. The system of claim 69 wherein that component is one or more of a field applicator or an acoustic transducer or acoustic standoff therefore.

71. The system of claim 1 wherein an acoustic transducer is coupled into a treatment subject one of a) through the skin or scalp, b) using an intermediate or interposed acoustic standoff, c) through a water or liquid bath, d) through a liquid or gel-filled membrane or balloon, or e) through a cooling or temperature control means.

72. The system of claim 1 wherein any of system software or hardware or the practitioner manages the temporal coordination of the acoustic applicator and the field applicator.

73. The system of claim 1 wherein the system contains or is designed to contain one or more of a) software, b) electronic circuitry, c) power supplies, d) logic, e) controllers, f) a display of any type, g) connecting cables or lumens, h) an I/O means, i) memory, j) patient data, k) treatment data, I) triggering inputs or outputs, m) a data recording or playback means, n) a data network interface, or o) any interface to another medical tool, including a medical tool used for imaging or neural functionality monitoring or recording.

74. The system of claim 1 wherein at least a portion of the system is worn or carried by the treatment subject and that allows some mobility of the patient.

75. The system of claim 1 wherein the system is capable of learning from or being adaptive to any one or more of the treatment subject, his/her neural activity, his/her response to treatment, or the detected performance of the system in any manner, neurally or physically.

76. The system of claim 1 wherein a targeted or localized neural modification of an at least temporary nature is applied to at least a patient's neural tissue portion for a diagnostic or therapeutic purpose by any one or more of:
(i) a cumulative or synergistic exposure of said neural tissue portion to both the acoustic energy and the applied field, said exposure being sequential, simultaneous, or near-simultaneous, said exposure to both causing an at least localized neural change;
(ii) a simultaneous or near-simultaneous exposure to said acoustic energy and energy field, said energy and field contributing to a real-time or near real-time interaction between the field and the tissue to produce an at least localized neural change; or
(iii) at least one of elements (i) and (ii) combined with a global or local pretreatment, preconditioning, cotreatment, post-treatment or post-conditioning delivered by one or more of any of an acoustic energy or field application before, during or after the application of (i) or (ii), the acoustic and/or field characteristics utilized in (iii) optionally being different from those used in (i) or (ii).

77. The system of claim 76 wherein said simultaneous or near-simultaneous exposure involves a vector cross-product physical interaction between the field and a vector related neural parameter, including a neural conductance path or conductance length.

78. The system of claim 76 wherein said sequential, simultaneous or near-simultaneous exposure involves a finely focused acoustic energy exposure and a coarser, less-focused or non-focused exposure to the field, said field optionally containing a field gradient.
